(19) United States
(12) Patent Application Publication
(10) Pub. No.: US 2026/0007508 A1
(43) Pub. Date: Jan. 8, 2026

(54) Soft Anchors and Methods of Tissue Repairs

(71) Applicant: Arthrex, Inc., Naples, FL (US)

(72) Inventors: Lane J. NORRIS, Naples, FL (US); Michael N. SCHMIDT, Naples, FL (US); John W. ANDREWS, Naples, FL (US)

(73) Assignee: Arthrex, Inc., Naples, FL (US)

(21) Appl. No.: 19/256,216

(22) Filed: Jul. 1, 2025

Related U.S. Application Data
(60) Provisional application No. 63/667,842, filed on Jul. 5, 2024.

(51) Int. Cl. A61F 2/08; A61B 17/00; A61B 17/04

(52) U.S. Cl. CPC A61F 2/0811; A61B 17/0401; A61B 2017/00862; A61B 2017/0409; A61B 2017/0445; A61B 2017/0462; A61B 2017/0464; A61F 2002/0852

(57) ABSTRACT

Surgical anchor constructs, fixation devices, and methods for tissue repairs are disclosed. A soft suture anchor has a cannulated sheath (that is fixed in bone) with dimensions designed to fit bone holes of about 1.0 to about 1.1 mm. At least one flexible strand is attached to the sheath and passed through the lumen of the sheath in a specific direction to form one or more adjustable, flexible, tensionable loops extending around and through the cannulated sheath.

### CROSS-REFERENCE TO RELATED APPLICATIONS

This application claim priority to U.S. Provisional Application No. 63/667,842 filed on Jul. 5, 2024, which is hereby incorporated by reference in its entirety herein.

### BACKGROUND

The disclosure relates to the field of surgery and, more specifically, to surgical suture constructs and methods of making, and tissue repairs for reconstructive surgeries.

### SUMMARY

Surgical flexible constructs, fixation devices, methods of making, and methods of tissue repairs are disclosed.

A surgical construct is a soft anchor with a sheath (that is fixed in bone) and at least one flexible strand attached to the sheath. A flexible strand can be suture that is passed through the lumen of the sheath in a specific direction. A soft anchor sheath can be a suture sheath. A sheath can have dimensions designed to fit bone holes of about 1.0 to about 1.1 mm. A surgical construct can create a self-tensioning, self-locking, reinforced repair. A surgical construct can be employed in knotted or knotless fixation of first tissue to second tissue, for example, fixation of soft tissue to bone.

Methods of tissue repairs are also disclosed. A surgical construct can provide tissue to tissue fixation with fewer passing steps, and with increased fixation and soft tissue compression. A tissue repair can be a knotted repair. A tissue repair can be a knotless repair.

### DESCRIPTION OF THE DRAWINGS

FIG. 1 illustrates a surgical construct.

FIG. 2 illustrates another surgical construct.

FIG. 3 illustrates an inserter assembled with the surgical construct of FIG. 2.

FIGS. 4-7 illustrate additional surgical constructs.

FIG. 8 illustrates an exemplary repair with surgical constructs.

FIG. 9 illustrates another exemplary repair with surgical constructs.

### DETAILED DESCRIPTION

The disclosure provides surgical fixation devices, constructs, methods for manufacturing, and tissue repairs and reconstructions.

A soft anchor construct includes a sheath and at least one flexible strand attached to the sheath. The at least one flexible strand can be suture that is passed through a lumen of the sheath in a specific direction to form at least one adjustable, flexible, tensionable loop through and around the sheath. A soft anchor construct can be a knotted construct. A soft anchor construct can be a knotless construct.

A soft anchor construct includes a cannulated sheath with a suture that passes through a lumen of the sheath and enters/exits the lumen of the sheath and forms at least one adjustable, flexible, tensionable loop. The at least one adjustable, flexible, tensionable loop can be knotted. The at least one adjustable, flexible, tensionable loop can be knotless.

The soft anchor sheath can consist essentially of suture. A suture sheath can have nano dimensions, i.e., dimensions that allow the sheath to fit bone holes of about 1.0 to about 1.1 mm. A surgical construct can create a self-tensioning, self-locking, reinforced repair. A surgical construct can be employed in knotless or knotted fixation of first tissue to second tissue, for example, fixation of soft tissue to bone.

Methods of tissue repairs with soft anchor constructs are also disclosed. An exemplary method includes inter alia the steps of: (i) passing at least one flexible strand through a lumen of a cannulated soft anchor sheath so that the at least one flexible strand enters/exits the lumen of the sheath and forms at least one adjustable, flexible, tensionable loop around and through the sheath; and (ii) employing the sheath for one or more tissue repair applications. The one or more tissue repair applications can include soft tissue repairs; orthopedic surgical repairs such as rotator cuff repairs, Achilles tendon repairs, patellar tendon repairs, ACL/PCL reconstructions, hip and shoulder reconstructions, and hand, wrist, foot, and ankle repairs, among many others. The one or more tissue repairs can be a knotted repair. The one or more tissue repairs can be a knotless repair.

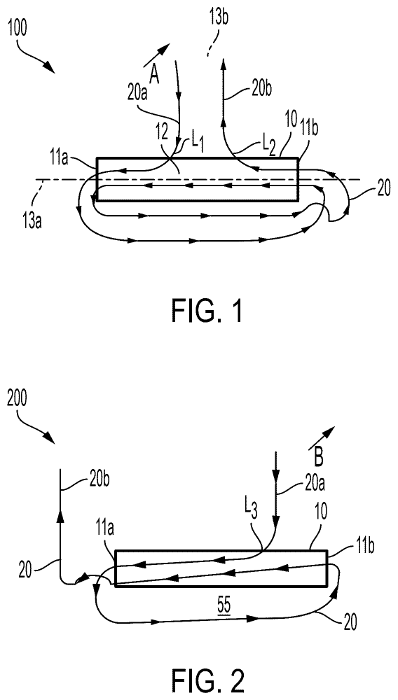

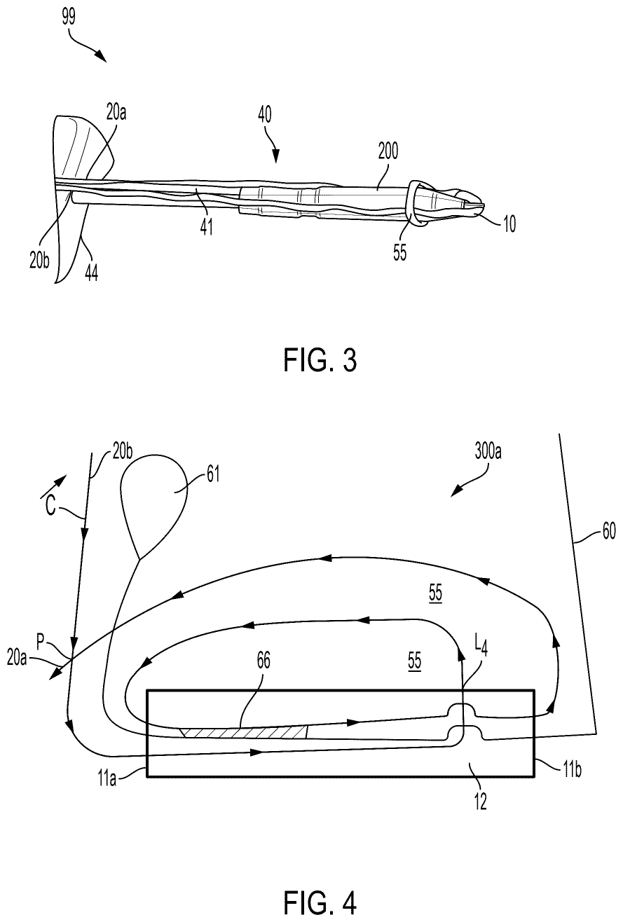

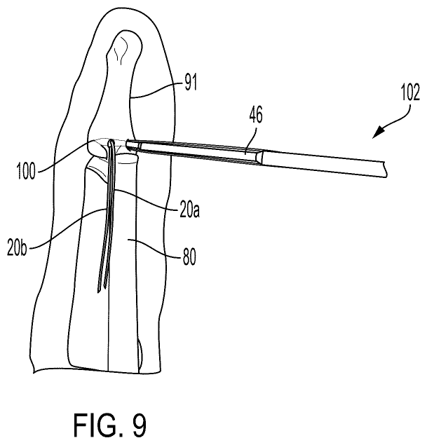

Referring now to the drawings, where like elements are designated by like reference numerals, FIGS. 1, 2 and 4-7 illustrate exemplary fixation devices 100, 200, 300a, 300b, 400a, 400b (soft anchors 100, 200, 300a, 300b, 400a, 400b; soft suture anchors 100, 200, 300a, 300b, 400a, 400b; all-suture anchors 100, 200, 300a, 300b, 400a, 400b; surgical constructs 100, 200, 300a, 300b, 400a, 400b; anchor constructs 100, 200, 300a, 300b, 400a, 400b; nano FiberTak® anchors 100, 200, 300a, 300b, 400a, 400b) of the present disclosure. FIG. 3 illustrates a surgical assembly 99 with exemplary fixation device 200. FIGS. 8 and 9 illustrate exemplary tissue repair 101, 102 with any of fixation devices 100, 200, 300a, 300b, 400a, 400b.

The fixation devices detailed below are soft anchors formed of “soft” materials, such as suture materials, that confer the ability to be inserted into bone sockets/holes/tunnels and bunch together, collapse, expand and/or change shape to fixate within the socket/hole/tunnel. In some embodiments, the soft anchor includes a cannulated sheath and one flexible strand attached to the sheath and forming at least one adjustable, flexible, tensionable loop. The flexible strand can be slidable relative to the sheath. In other embodiments, the soft anchor includes a sheath and a plurality of flexible strands, wherein some or all of the flexible strands are slideable relative to the sheath and forming at least one adjustable, flexible, tensionable loop. The soft anchors can be utilized in various surgical techniques to attach tissue to bone. As detailed below, the adjustable, flexible, tensionable loops can form a knotted construct or a knotless construct.

FIG. 1 illustrates exemplary soft anchor 100 including cannulated sheath 10 and flexible strand 20. Sheath 10 can be in the form of a tubular sleeve or tubular member made of a flexible material, such as a braided, woven, or knitted structure made of yarns, fibers, filaments, sutures or similar materials, or combinations of these materials. In one embodiment, sheath 10 is constructed of ultra-high molecular weight polyethylene (UHMWPE). In one embodiment, sheath 10 is constructed of UHMWPE and polyester. In another embodiment, sheath 10 is constructed of UHMWPE with an elastic component. In another embodiment, sheath 10 is constructed of polyester suture material with an elastic component. The elastic component can be elastane. The elastic component can be incorporated into the sheath via braiding, weaving, and/or knitting. In an embodiment, an elastic sheath can include a combination of elastic, polyester, and UHMWPE all braided in a tubular jacket. In an embodiment, an elastic sheath can consist essentially of elastane, polyester, and UHMWPE. The elastic component provides elasticity while the other components provide strength and limit the elongation of the suture.

Sheath 10 can also be any tubular or non-tubular structure having a lumen 12 along its length. The lumen can have a constant diameter. The lumen can have a varying diameter along its length. Sheath 10 can have nano dimensions, i.e., dimensions that allow the sheath to fit very small bone holes or openings of about 1.0 to about 1.1 mm. In exemplary embodiments, sheath 10 can have a length of less than about 10 mm, more preferably less than about 5 mm, i.e., very small dimensions that allow the implant/sheath to fit bone holes of minute sizes such as about 1.0 to about 1.1 mm.

Soft anchor 100 can include at least one flexible strand 20 passing through cannulated sheath 10. Sheath 10 includes a tubular body that extends between opposing ends 11a, 11b. The opposing ends 11a, 11b can be open or closed ends. The tubular body establishes a bore or lumen 12 that extends between the opposing ends 11a, 11b. As shown in FIG. 1, one or more flexible strands 20 (flexible coupler 20; suture 20) passes through at least a portion of bore of the sheath 10 in a specific manner, i.e., in the direction of arrow A.

In an exemplary-only embodiment and as depicted in FIG. 1, flexible strand 20 enters the sheath 20 at a location L1 (situated between the opposing ends 11a, 11b); passes through at least a portion of lumen 12 of sheath 10; exits one of the opposing ends 11a, 11b (for example, end 11a); enters the other of the opposing ends 11a, 11b (for example, end 11b); passes through the lumen 12 of sheath 10; exits the one of the opposing ends 11a, 11b (for example, end 11a); re-enters the other of the opposing ends 11a, 11b (for example, end 11b); passes through at least a portion of the lumen 12 of sheath 10; and exits the lumen 12 and sheath 10 at a location L2 (situated between the point L1 and one of the opposing ends 11a, 11b), forming at least one adjustable, flexible, knotless, tensionable loop 55.

Locations L1, L2 can be points of entry and/or exit in the form of openings formed through the sheath 10. Locations L1, L2 can be pre-formed openings formed within and through the sheath 10 during the manufacturing process. Locations L1, L2 can be formed in situ, i.e., during the tissue repair by simply passing the flexible strand 20 through the lumen 12 of the sheath and entering and exiting the sheath at the specific locations.

In an embodiment, and as shown in FIG. 1, locations L1, L2 are located about symmetric relative to transversal axis 13b of the sheath 10 and on a same side relative to longitudinal axis 13a of the sheath 10. In an embodiment, locations L1, L2 can be located asymmetric relative to transversal axis 13b of the sheath 10 and on a same side relative to longitudinal axis 13a of the sheath 10. In an embodiment, locations L1, L2 can be located about symmetric relative to transversal axis 13b of the sheath 10 and on different sides relative to longitudinal axis 13a of the sheath 10. In an embodiment, locations L1, L2 can be located asymmetric relative to transversal axis 13b of the sheath 10 and on different sides relative to longitudinal axis 13a of the sheath 10.

Flexible strand 20 can assist in bunching together the sheath 10 once the soft anchor 100 is inserted into bone or secured over bone and once ends 20a, 20b the flexible strand 20 are tensioned. Ends 20a, 20b can be employed in additional fixation steps and/or can be knotted.

Reference is now made to FIG. 2 which illustrates soft anchor 200 which is about similar to soft anchor 100 detailed above in that soft anchor 200 also include a cannulated sheath 10 with at least one flexible strand 20 passing through cannulated sheath 10 to form at least one adjustable, flexible, tensionable loop 55. However, soft anchor 200 differs from soft anchor 100 in the location of the entry and exit points of the flexible strand 20.

As shown in FIG. 2, one or more flexible strands 20 (flexible coupler 20; suture 20) passes through at least a portion of bore of the sheath 10 in a specific manner, i.e., in the direction of arrow B. In an exemplary-only embodiment and as depicted in FIG. 2, flexible strand 20 enters the sheath 20 at a location L3 (situated between the opposing ends 11a, 11b); passes through at least a portion of lumen 12 of sheath 10; exits one of the opposing ends 11a, 11b (for example, end 11a); re-enters the other of the opposing ends 11a, 11b (for example, end 11b); passes through lumen 12 of sheath 10; and exits the lumen 12 and sheath 10 at the one of the opposing ends 11a, 11b (for example, end 11a), forming at least one adjustable, flexible, knotless, tensionable loop 55.

Locations L3 can be a point of entry and/or exit in the form of an opening formed through the sheath 10. Location L3 can be a pre-formed opening formed within and through the sheath 10 during the manufacturing process. Location L3 can be formed in situ, i.e., during the tissue repair by simply passing the flexible strand 20 through the sheath 10 to enter the lumen 12 at the specific location L3.

In an embodiment, and as shown in FIG. 2, location L3 is located closer to opposing end 11b, i.e., the end opposite end 11a where the flexible strand 20 exits the sheath 10.

Flexible strand 20 can assist in bunching together the sheath 10 once the soft anchor 100, 200 is inserted into bone or secured over bone and once ends 20a, 20b the flexible strand 20 are tensioned. Flexible strand 20 can be of any length and can include one or more free ends 20a, 20b that extend outside of the sheath 10. Additional strands can be passed through the tubular body of sheath 10, for example, suture passers and/or additional filaments and/or flexible couplers, in same or different orientations, as desired and depending on the specific surgical procedure to be conducted. In additional embodiments, flexible strand 20 can enter and exit the sheath multiple times, at same or different locations to form a plurality of loops 55. Ends 20a, 20b can be knotted and/or employed in additional fixation steps.

FIG. 3 illustrates exemplary soft anchor 200 loaded onto inserter 40 to form assembly 99. Ends 20a, 20b of flexible strand 20 can be secured to handle 44 of inserter 40. Loop 55 extends in a direction about perpendicular to a longitudinal axis of the inserter shaft 41.

Reference is now made to FIGS. 4-7 which illustrate additional exemplary soft anchors 300a, 300b, 400a, 400b. Soft anchors 300a, 300b, 400a, 400b are about similar to soft anchors 100, 200 detailed above in that these anchors also include a cannulated sheath 10 with at least one flexible strand 20 passing through cannulated sheath 10 to form at least one adjustable, flexible, tensionable loop 55 (double-wrapped around sheath 10). However, soft anchors 300a, 300b, 400a, 400b are knotless constructs and not knotted constructs as soft anchors 100, 200, detailed above. As detailed below, one end of the repair suture 20 (end 20a) passes through and around sheath 10 in a specific manner to form at least two adjustable, flexible, knotless, tensionable loop 55; the other end of repair suture 20 (end 20b) is pierced by end 20a and then passed through repair suture 20 to form a splice within the sheath 10.

To form soft anchor 300a, and as shown in FIG. 4, one or more flexible strands 20 (flexible coupler 20; suture 20) passes through at least a portion of bore 12 of the sheath 10 in a specific manner, i.e., in the direction of arrow C. In an exemplary-only embodiment and as depicted in FIG. 4, end 20a of flexible strand 20 enters the sheath 10 at one of the ends 11a, 11b (for example, end 11a); passes through at least a portion of lumen 12 of sheath 10; exits the lumen 12 and the sheath 10 at location L4; re-enters the sheath 10 through same end 11a; passes through lumen 12 of sheath 10; and exits the lumen 12 and sheath 10 at an end opposed the entrance end (for example, end 11b); and pierces end 20b of the flexible strand 20 at location P, forming at least one adjustable, flexible, knotless, tensionable loop 55. In the embodiment of FIG. 4, flexible strand 20 forms two adjustable, flexible, knotless, tensionable loop 55. End 20a of flexible strand 20 is secured to end 20b by being glued or affixed to it, or by other securing methods known in the art.

A suture passing device 60 (shuttling device 60; suture passer 60) such as a Nitinol loop is attached to flexible strand 20 as shown in FIG. 4, to aid in the splice formation. Once adjustable, flexible, knotless, tensionable loops 55 have been formed, end 20b of flexible strand 20 is passed through loop 61 of the suture passer 60. Suture passer 60 is pulled out of sheath 10 to pull the repair suture 20 through itself at splice 66, and to create a cinching loop around soft tissue to be attached to bone (in a manner similar to a knotless soft anchor) as part of a knotless repair.

Soft anchor 300b of FIG. 6 is about similar to soft anchor 300a detailed above with reference to FIG. 4 but differs in the location of suture passer 60 attached to flexible strand 20 and subsequent formation of the splice. In soft anchor 300a, suture passer 60 is part of a top loop 55 (second formed loop 55) so that splice 66 is part of the top loop 55. In soft anchor 300b, suture passer 60 is part of a bottom loop 55 (first formed loop 55) so that splice 66 is part of the bottom loop 55.

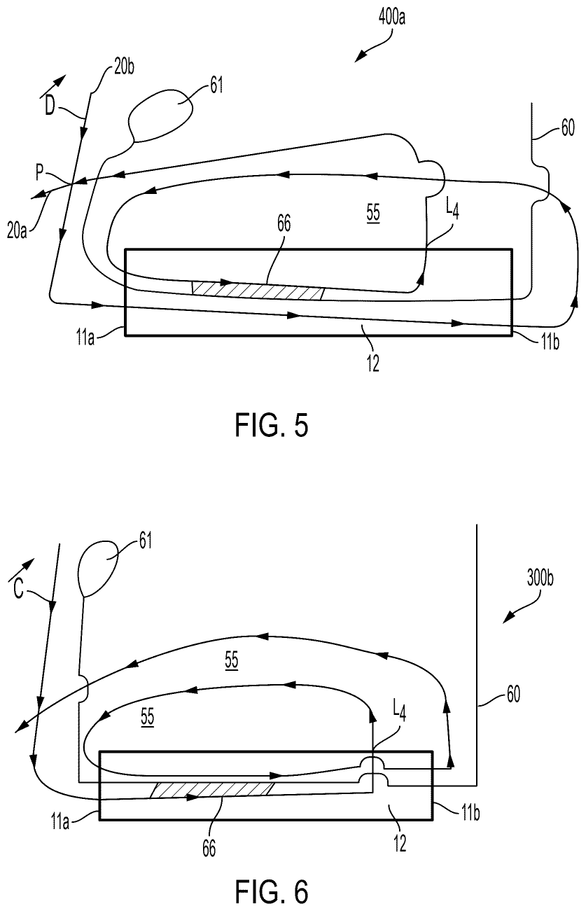

Reference is now made to FIG. 5, which illustrates soft anchor 400a, which is also a knotless fixation device. As shown in FIG. 5, flexible strand 20 (flexible coupler 20; suture 20) passes through at least a portion of bore 12 of the sheath 10 in a specific manner, i.e., in the direction of arrow D. In an exemplary-only embodiment, end 20a of flexible strand 20 enters the sheath 20 at one of the ends 11a, 11b (for example, end 11a); passes through lumen 12 of sheath 10; exits the lumen 12 and the sheath 10 at an opposing end of the sheath 10 (for example, end 11b); re-enters the sheath through same end 11a; passes through a portion of lumen 12 of sheath 10; exits the lumen 12 and sheath 10 at location L4; and pierces end 20b of the flexible strand 20 at location P, forming at least one adjustable, flexible, knotless, tensionable loop 55. In the embodiment of FIG. 5, flexible strand 20 forms two adjustable, flexible, knotless, tensionable loops 55. End 20a of flexible strand 20 is secured to end 20b by being glued or affixed to it, or secured by other methods known in the art.

A suture passing device 60 such as a Nitinol loop is attached to flexible strand 20 as shown in FIG. 5. Once adjustable, flexible, knotless, tensionable loops 55 have been formed, remaining end 20b of flexible strand 20 is passed through loop 61 of the suture passer 60, to pull the repair suture 20 through itself at splice 66, and to create a cinching loop around soft tissue to be attached to bone (in a manner similar to a knotless soft anchor) as part of a knotless repair.

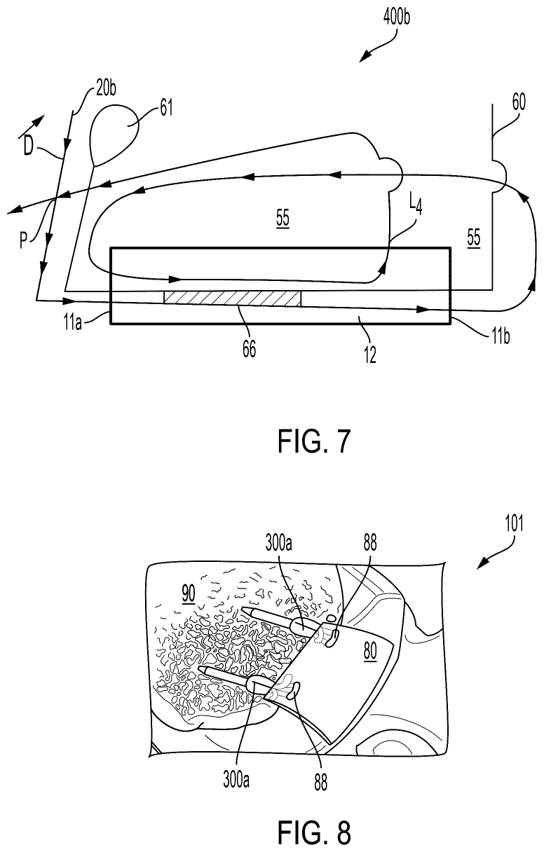

Soft anchor 400b of FIG. 7 is about similar to soft anchor 400a detailed above with reference to FIG. 5 but differs in the location of the attachment of suture passer 60 to flexible strand 20. In soft anchor 400a, suture passer 60 is part of a top loop 55 (second formed loop 55) of flexible strand 20 so that splice 66 is part of the top loop 55. In soft anchor 400b, suture passer 60 is part of a bottom loop 55 (first formed loop 55) of flexible strand 20 so that splice 66 is part of the bottom loop 55.

Location L4 can be a point of entry and/or exit in the form of an opening formed through the sheath 10. In an embodiment, and as shown in FIGS. 4-7, location L4 is located closer to opposing end 11b, i.e., the end opposite end 11a where the flexible strand 20 first enters the sheath 10.

Flexible strand 20 can be a suture. Non-limiting examples of suitable sutures include FiberWire®, TigerWire®, or FiberChain® suture, although any type of suture may be utilized, including cored or coreless sutures. In another embodiment, flexible strand 20 can be suture tape, such as FiberTape®. Flexible strand 20 can include any soft, flexible strand of material.

Soft anchor 100, 200, 300a, 300b, 400a, 400b is configured for use in various soft tissue repairs or fixations and can be fixated inside bone or over bone for attaching tissue (e.g., ligament, tendon, graft, etc.) to bone. For example, the soft anchor 100, 200, 300a, 300b, 400a, 400b can be used in conjunction with a variety of orthopedic surgical repairs, including but not limited to rotator cuff repairs, Achilles tendon repairs, patellar tendon repairs, ACL/PCL reconstructions, hip and shoulder reconstructions, among many others. The fixation can be on or over bone. Particular applications of soft anchor 100, 200, 300a, 300b, 400a, 400b are repairs of hand, wrist, foot, ankle and small joints for distal extremities, given the nano dimensions of the soft anchors.

Soft anchor 100, 200, 300a, 300b, 400a, 400b is a “soft” construct because it is formed of soft materials such as yarns, fibers, filaments, strings, fibrils, strands, sutures, etc., or any combination of such materials. The soft materials may be synthetic or natural materials, or combinations of synthetic and natural materials, and may be bio-degradable or non-degradable, and may be elastic or non-elastic within the scope of this disclosure. In one non-limiting embodiment, the soft anchor 100, 200, 300a, 300b, 400a, 400b is made exclusively of soft, suture-based materials. Soft anchor 100, 200, 300a, 300b, 400a, 400b can be an “all-suture” construct.

Soft anchor 100, 200, 300a, 300b, 400a, 400b can be manufactured via braiding weaving and/or knitting processes, or combination of these processes, with or without an elastic component in them, and with a variable pick count throughout that allows the soft anchor 100, 200, 300a, 300b, 400a, 400b to deform according to the pick count.

FIG. 8 illustrates an exemplary repair 101 (ankle ligament reconstruction) with any of soft anchors 100, 200, 300a, 300b, 400a, 400b detailed above. A drill guide (for example, a knotless drill guide) and a K-wire (such as a 1.8 mm K-wire) can be employed to create a bone tunnel about 1 cm from the distal tip of fibula 90. Keeping the guide in place, a soft anchor 100, 200, 300a, 300b, 400a, 400b can be inserted.

For an exemplary knotless lateral ankle ligament reconstruction, repair suture 20 of exemplary-only soft anchor 300a is passed through soft tissue 80 (talofibular ligament 80) and then through a loop of the suture passer (such as loop 61) attached to the flexible strand 20. By pulling the suture passer out of sheath 10, the suture 20 is pulled through itself at splice 66 to form a cinching loop 88 around soft tissue 80 to be attached to bone 90. The steps can be repeated with a second anchor (either knotted or knotless anchor) for final fixation. Repair 101 is a knotless repair. Sheath 10 is secured within and against bone 90 so that the sheath material allows bunching and deformation within the bone hole, socket, or tunnel, allowing the sheath to anchor in bone 90 and to increase the overall fixation. Flexible coupler 20 can be passed through or around soft tissue 80 to secure it to bone 90 by forming at least one loop 88 around soft tissue 80.

FIG. 9 illustrates repair 102 which is an avulsion fracture repair typically involving a piece of bone attached to a ligament or tendon breaking away from the main part of the bone. Repair 102 is an exemplary FDP (flexor digitorum profundus) avulsion repair wherein reconstruction of the FDP is usually performed using free tendon graft due to the retraction of tendon ends and shortening of the tendon.

As shown in FIG. 9, at least one (typically two) of soft anchor 100, 200, 300a, 300b, 400a, 400b are placed into previously drilled holes in distal phalanx 91 with an inserter instrument (such as inserter 40 or another inserter 46). Ends 20a, 20b of flexible strand 20 can be used to perform a modified Becker or an alternative repair of surgeon's preference, and the tendon 80 is brought back to bone. The repair can be supplemented by suturing the FDP remnant back over the top of the repair site using a flexible coupler such as FiberWire® suture.

Flexible coupler 20 of tissue repairs 101, 102 can be employed for attachment of soft tissue 80 to bone 90, 91 by passing the flexible coupler through or around soft tissue and forming at least one adjustable, flexible, continuous, tensionable loop (such as cinching knotless 88) around or through soft tissue 80. One or more flexible couplers 20 and optional shuttling strands may extend through the lumen 12 of sleeve 10 in similar or different directions and/or orientations and/or locations. The flexible tubular sleeve 10 with the flexible couplers and shuttling strands may be secured into or onto bone, and flexible strands can pass over soft tissue (rotator cuff) and are secured into bone to approximate soft tissue to bone. Details of an exemplary soft suture anchor with a soft anchor sleeve (sheath or tubular member) and flexible shuttling strands are set forth, for example, in U.S. Pat. No. 10,849,734 issued Dec. 1, 2020, entitled “Methods of Tissue Repairs,” the disclosure of which is incorporated by reference in its entirety herein.

Flexible coupler 20 can be further attached to one or more fixation devices such as any anchors, for example, knotted anchors, knotless anchors, or all-suture anchors, or any devices that confer secure attachment and fixation of soft tissue 80 over bone 90. The fixation device can be a knotless anchor such as a two-piece Arthrex PushLock® anchor, disclosed in U.S. Pat. No. 7,329,272, or an Arthrex SwiveLock® anchor, disclosed in U.S. Pat. Nos. 8,012,174 and 9,005,246, the disclosures of both of which are fully incorporated by reference in their entirety herein. The fixation device can also be another all-suture soft anchor like soft anchors 100, 200, 300a, 300b, 400a, 400b detailed above. Flexible coupler 20 can consist essentially of elastic suture.

A method of tissue reconstruction 101, 102 comprises attaching soft tissue 80 to bone 90, 91 with at least one soft anchor 100, 200, 300a, 300b, 400a, 400b. The method can include forming at least one adjustable, flexible, tensionable loop 55 by passing flexible coupler 20 through sheath 10 in a direction of arrow A, B, C, D; and securing the soft anchor 100, 200, 300a, 300b, 400a, 400b into bone 90. The method can also include passing the at least one flexible coupler 20 through or around soft tissue 80 to attach the soft tissue to bone. The method can further include forming, with the at least one flexible coupler 20, at least one adjustable, flexible, continuous, tensionable loop 88 around or through soft tissue 80. The method can further include securing the at least one flexible coupler 20 to a fixation device. The fixation device can be a knotless anchor. The fixation device can be a hard body anchor. The fixation device can be a soft anchor. The at least one flexible coupler 20 can consist essentially of elastic suture. The soft tissue 80 can be part of a Brostrom repair. A plurality of soft anchors 100, 200, 300a, 300b, 400a, 400b can be used for a soft tissue repair, such as a small joint repair. The repair can be an avulsion repair.

The constructs and methods of the present disclosure provide increased fixation and consistent longer lasting longer pressure on the tissue.

The constructs of the present disclosure have applicability to any tissue repair and surgical procedure such as, for example, small joint repairs, rotator cuff repairs, Achilles tendon repair, patellar tendon repair, ACL/PCL reconstruction, hip and shoulder reconstruction procedures, and applications for elastic suture used in or with suture anchors. The surgical constructs and repair methods of the present disclosure can be employed in tissue repairs that are knotted or knotless.

The surgical constructs and repair methods of the present disclosure can be employed in tissue repairs that do not involve knot tying, for example, for use with suture anchors (such as PushLock® and/or SwiveLock® suture anchors) or for knotless arthroscopic suture repairs (such as knotless single row rotator cuff repair, or SpeedBridge™ repairs using no knots and only suture passing steps), among many others.

An exemplary SutureBridge™ tendon repair technique, developed by Arthrex, Inc., and disclosed in U.S. Pat. No. 8,012,174 (the disclosure of which is herein incorporated by reference in its entirety) consists of a tied medial row constructed with two threaded suture anchors, combined with knotless lateral fixation using two Arthrex PushLocks® constructs. The construct enhances footprint compression and promotes tendon healing-to-bone with minimal knot tying.

An exemplary SpeedBridge™ technique, also developed by Arthrex, Inc., and disclosed in U.S. Pat. No. 9,005,246 (the entire disclosure of which is herein incorporated by reference) uses a threaded swivel anchor which can be combined with any of constructs 100, 200 to create a quick and secure SutureBridge™ construct with no knots and only two suture passing steps.

A method of soft tissue reconstruction comprises attaching soft tissue 80 to bone 90 with at least one surgical construct 100, 200, 300a, 300b, 400a, 400b. The method can further comprise securing the surgical construct 100, 200, 300a, 300b, 400a, 400b to bone 90; and securing at least one flexible coupler 20 to soft tissue 80. The surgical construct 100, 200, 300a, 300b, 400a, 400b can be a unitary, one-piece construct comprising elastic material. The flexible coupler 20 can comprise elastic material. The method can further comprise attaching flexible coupler 20 to a fixation device. The fixation device can be a knotless or knotted anchor.

Flexible coupler 20 can be formed of a high strength suture material such as FiberWire® suture, sold by Arthrex, Inc. of Naples, Fla., and described in U.S. Pat. No. 6,716,234, the disclosure of which is incorporated by reference herein. FiberWire® suture is formed of an advanced, high-strength fiber material, namely ultrahigh molecular weight polyethylene (UHMWPE), sold under the tradenames Spectra® (Honeywell International Inc., Colonial Heights, Va.) and Dyneema® (DSM N.V., Heerlen, the Netherlands), braided optionally with at least one other elastic fiber, natural or synthetic, to form lengths of suture material. Flexible coupler 20 can also include suture tape such as FiberTape® suture tape (as disclosed in U.S. Pat. No. 7,892,256, the disclosure of which is incorporated in its entirety herewith) or collagen tape, or wide “tape like” material, or combinations thereof.

Flexible coupler 20 can consist essentially of suture material and elastane, or combination of suture material and elastane and other materials such as long chain synthetic polymers like polyester and nylon, or materials such as PET, silk nylon or absorbable polymers, or coating materials (such as wax, silk, or silicone products), among many others. Flexible coupler 20 can consist of strands with cross-sections of various forms and geometries, including round, oval, rectangular, or flat, among others, or combinations of such forms and geometries. In an embodiment, at least one flexible coupler 20 can be provided as a suture which is braided, knitted or woven.

Parts or all of soft anchor 100, 200, 300a, 300b, 400a, 400b including sheath 10 and flexible coupler 20 can be also coated and/or provided in different colors. In an embodiment, parts (or all) of sheath 10 and flexible coupler 20 can be coated (partially or totally) with wax (beeswax, petroleum wax, polyethylene wax, or others), silicone (Dow Corning silicone fluid 202A or others), silicone rubbers (Nusil Med 2245, Nusil Med 2174 with a bonding catalyst, or others) PTFE (Teflon, Hostaflon, or others), PBA (polybutylate acid), ethyl cellulose (Filodel) or other coatings, to improve lubricity of the construct, pliability, handleability or abrasion resistance, for example.

Parts or all of sheath 10 and/or flexible coupler 20 can be also provided with tinted tracing strands, or otherwise contrast visually with other parts of the construct, which remain a plain, solid color, or displays a different tracing pattern, for example. Various structural elements of the surgical constructs may be visually coded, making identification and handling of the suture legs simpler. Easy identification of suture in situ is advantageous in surgical procedures.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed is:

1. A soft anchor comprising:
a cannulated sheath having a length, a first open end, and a second open end; and
a flexible coupler attached to the cannulated sheath and forming a at least one adjustable, flexible, tensionable loop around the cannulated sheath.

2. The soft anchor of claim 1, wherein the length of the cannulated sheath is configured to fit a bone hole of about 1.0 to about 1.1 mm.

3. The soft anchor of claim 1, wherein the flexible coupler enters the cannulated sheath at a first location, extends within the cannulated sheath, exits the cannulated sheath at the first open end, enters the cannulated sheath though the second open end, extends within the cannulated sheath, exits the cannulated sheath at the first open end, re-enters the cannulated sheath though the second open end, extends within the cannulated sheath, and exits the cannulated sheath at a second location, wherein the second location is different from the first location.

4. The soft anchor of claim 3, wherein the first location and the second location are about symmetrically oriented relative to a transversal axis of the cannulated sheath.

5. The soft anchor of claim 3, wherein the first location and the second location are each spaced apart from the first open end and the second open end of the cannulated sheath.

6. The soft anchor of claim 1, wherein the flexible coupler enters the cannulated sheath at a first location, extends within the cannulated sheath, exits the cannulated sheath at the first open end, enters the cannulated sheath though the second open end, extends within the cannulated sheath, and exits the cannulated sheath through the first open end.

7. The soft anchor of claim 6, wherein the first location is spaced apart from the first open end and the second open end.

8. The soft anchor of claim 1, wherein at least one region of the flexible coupler is attached to a fixation device.

9. The soft anchor of claim 8, wherein the fixation device is a knotless suture anchor.

10. The soft anchor of claim 1, wherein the soft anchor is an all-suture anchor, and the flexible coupler is round suture.

11. The soft anchor of claim 1, wherein the cannulated sheath is elastic.

12. The soft anchor of claim 1, wherein the soft anchor is manufactured as a one-piece construct.

13. The soft anchor of claim 1, wherein the flexible coupler forms two adjustable, flexible, tensionable loops and wherein the soft anchor is a knotless construct.

14. The soft anchor of claim 1, wherein the flexible coupler forms one or two adjustable, flexible, tensionable loops and wherein the soft anchor is a knotted construct.

15. A method of soft tissue repair comprising:
attaching at least one flexible coupler to a soft anchor sheath by passing the at least one flexible coupler through a lumen of the soft anchor sheath to form at least one adjustable, flexible, tensionable loop around the anchor sheath;
securing the soft anchor sheath into bone; and
securing ends of the at least one flexible coupler to soft tissue to attach the soft tissue to the bone.

16. The method of claim 15, further comprising:
passing the at least one flexible coupler through the soft anchor sheath at a first location;
extending the at least one flexible coupler through the lumen of the soft anchor sheath;
exiting a first open end of the soft anchor sheath;
entering a second open end of the soft anchor sheath;
extending the at least one flexible coupler through the lumen of the soft anchor sheath;
exiting the first open end of the soft anchor sheath;
re-entering the second open end of the soft anchor sheath; and
exiting the soft anchor sheath by passing the at least one flexible coupler through the soft anchor sheath at a second location.

17. The method of claim 16, wherein the first location is separate from the second location.

18. The method of claim 16, wherein each of the first location and the second location is between the first open end and the second open end.

19. The method of claim 15, further comprising:
passing the at least one flexible coupler through the soft anchor sheath at a first location;
extending the at least one flexible coupler through the lumen of the soft anchor sheath;
exiting a first open end of the soft anchor sheath;
entering a second open end of the soft anchor sheath;
extending the at least one flexible coupler through the lumen of the soft anchor sheath; and
exiting the soft anchor sheath by passing the at least one flexible coupler through the first open end.

20. The method of claim 19, wherein the first location is between the first open end and the second open end.

21. The method of claim 15, further comprising securing the soft anchor sheath to a tip of an inserter so that one of the at least one adjustable, flexible, tensionable loop extends about perpendicular to a longitudinal axis of the inserter.

22. The method of claim 15, wherein the soft tissue repair is a foot, ankle, hand or wrist repair.

23. The method of claim 15, wherein the soft tissue repair is a Bronstrom repair.

24. The method of claim 15, wherein the soft tissue repair is an avulsion repair.

25. The method of claim 15, wherein the soft tissue repair is a knotted repair.

26. The method of claim 15, wherein the soft tissue repair is a knotless repair.
